(19) United States
(12) Patent Application Publication
(10) Pub. No.: US 2012/0022294 A1
(43) Pub. Date: Jan. 26, 2012

(54) Axomadol or a Metabolite Thereof for Use in the Treatment of Irritable Bowel Syndrome

(75) Inventors: Klaus Schiene, Juechen (DE); Petra Bloms-Funke, Wuerselen (DE); Thomas Christoph, Aachen (DE); Wolfgang Schroeder, Aachen (DE)

(73) Assignee: Gruenenthal GmbH, Aachen (DE)

(21) Appl. No.: 13/172,281

(22) Filed: Jun. 29, 2011

Related U.S. Application Data
(60) Provisional application No. 61/360,209, filed on Jun. 30, 2010.

(30) Foreign Application Priority Data
Jun. 30, 2010 (EP) EP 10 006 781.8

(51) Int. Cl. C07C 215/42

(52) U.S. Cl. 564/443

(57) ABSTRACT

The invention relates to axomadol or a metabolite thereof for use in the treatment of irritable bowel syndrome.

The invention relates to axomadol or a metabolite thereof for use in the treatment of irritable bowel syndrome.

Irritable bowel syndrome (IBS or spastic colon) is characterized by abdominal pain and/or discomfort related to abnormal bowel habits. It is probably the most common disorder encountered by gastroenterologists and also the most common gastrointestinal disorder seen in primary care. In the Western world, IBS appears to affect up to 20% of the population at any given time, although the prevalence figures vary substantially depending on the definition of IBS (cf. Posserud I, Ersryd A, Simrén M, Functional findings in irritable bowel syndrome. World J. Gastroenterol. 2006; 12(18):2830-2838). Due to its high prevalence and, for many patients, chronic nature and incapacitating symptoms the cost of IBS to society is substantial. The pathophysiology of IBS is complex and still incompletely known. Both central and peripheral factors, including psychosocial factors, abnormal gastrointestinal (GI) motility and secretion, visceral hypersensitivity and referred pain, are thought to contribute to the symptoms of IBS. Validated schemata for irritable bowel syndrome are available such as the Manning criteria and the Rome criteria that allow for the diagnosis of irritable bowel syndrome to be made based upon the history of the patient. The subclassification of IBS is based on the predominant symptom of diarrhea (IBS with predominant diarrhea, IBS-D), constipation (IBS with predominant constipation, IBS-C) or mixed symptoms (IBS with alternating constipation and diarrhea, IBS-C)(Grundmann O, Yoon S L, Irritable bowel syndrome: epidemiology, diagnosis and treatment: an update for health-care practitioners; J. Gastroenterol. Hepatol., 2010; 25(4):691-699). Due to the limited efficacy and tolerability of current treatment, there is still a great need to find new treatment alternatives for this big patients group.

It was an object of the invention to provide a compound for use in the treatment of irritable bowel syndrome, which preferably has advantages over other active substances known from the prior art.

This object is achieved by the subject matter of the claims.

The invention relates to axomadol or a metabolite thereof for use in the treatment of irritable bowel syndrome. Preferably the disorder to be treated is selected from the group consisting of irritable bowel syndrome with diarrhea, diarrhea-predominant irritable bowel syndrome, irritable bowel syndrome without diarrhea, constipation-predominant irritable bowel syndrome, irritable bowel syndrome with alternating stool pattern (irritable bowel syndrome with alternating constipation and diarrhea, mixed irritable bowel syndrome) and post infectious irritable bowel syndrome.

Axomadol, i.e. (1RS,3RS,6RS)-6-dimethylaminomethyl-1-(3-methoxyphenyl)-cyclohexane-1,3-diol, is a synthetic, centrally active analgesic, which is effective in the treatment of moderate to severe, acute or chronic pain. Axomadol may be present in form of the free base, or in form of a salt, or a solvate.

For the purposes of the present invention axomadol includes (1RS,3RS,6RS)-6-dimethylaminomethyl-1-(3-methoxyphenyl)-cyclohexane-1,3-diol and its individual enantiomers, (+)-axomadol and (−)-axomadol, as well as physiologically acceptable salts and solvates thereof.

It was further discovered that metabolites of axomadol such as O-demethyl axomadol (6-dimethylaminomethyl-1-(3-hydroxyphenyl)-cyclohexane-1,3-diol) and its stereoisomers, particularly its enantiomers, more particularly (+)-O-demethyl axomadol, as well as physiologically acceptable salts and solvates thereof are also useful for the treatment of irritable bowel syndrome. The synthesis of axomadol and O-demethyl axomadol are disclosed, for example, in EP 0 753 506 A1.

Other metabolites of axomadol that might be useful for the treatment of irritable bowel syndrome are N-demethyl axomadol (6-monomethylaminomethyl-1-(3-methoxyphenyl)-cyclohexane-1,3-diol) and N,O-didemethyl axomadol (6-monomethylaminomethyl-1-(3-hydroxyphenyl)-cyclohexane-1,3-diol) and their respective stereoisomers, particularly their enantiomers, more particularly (+)-N-demethyl axomadol and (+)-N,O-didemethyl axomadol as well as physiologically acceptable salts and solvates thereof. These metabolites may be obtained according to methods well-known to those skilled in the art.

Thus, in another aspect the present invention also relates to metabolites of axomadol selected from the group consisting of (±)-N-demethyl axomadol, (+)-N-demethyl axomadol, (−)-N-demethyl axomadol, (±)-N,O-didemethyl axomadol, (+)-N,O-didemethyl axomadol and (−)-N,O-didemethyl axomadol as well as physiologically acceptable salts and solvates thereof.

If a metabolite of axomadol is used according to the present invention the use of (+)-O-demethyl axomadol or a physiologically acceptable salt thereof is particularly preferred.

Suitable pharmaceutically acceptable salts include salts of inorganic and/or organic acids such as e.g. acetic acid, 2,2-dichloroacetic acid, acylated amino acids, preferably acetylated amino acids such as e.g. N-acetylalanine, N-acetylcysteine, N-acetylglycine, N-acetylisoleucine, N-acetylleucine, N-acetylmethionine, N-acetylphenylalanine, N-acetylproline, N-acetylserine, N-acetylthreonine, N-acetyltyrosine, N-acetylvaline, adipic acid, alginic acid, ascorbic acid, L-aspartic acid, benzenesulphonic acid, benzoic acid, 4-acetamidobenzoic acid, (+)-camphoric acid, (−)-camphoric acid, (+)-camphor sulphonic acid, (−)-camphor sulphonic acid, (+)-camphor-10-sulphonic acid, (−)-camphor-10-sulphonic acid, (±)-camphor-10-sulphonic acid, capric acid, caproic acid, caprylic acid, cation exchange resins, cinnamic acid, citric acid, cyclohexyl sulphamic acid, sulphuric acid monododecyl ester, ethane-1,2-sulphonic acid, ethanesulphonic acid, 2-hydroxyethanesulphonic acid, formic acid, fumaric acid, mucic acid (galactosaccharic acid), gentisic acid, glucose monocarboxylic acid (glucoheptonic acid), d-gluconic acid, d-glucuronic acid, L-glutamic acid, α-oxoglutaric acid (α-ketoglutaric acid), hydroxyacetic acid (glycollic acid), hippuric acid (N-benzoylglycine), hydrogen bromide, hydrogen chloride, (+)-L-lactic acid, (±)-DL-lactic acid, lactobionic acid (4-O-β-D-galactopyranosyl-D-gluconic acid), maleic acid, (−)-L-malic acid, malonic acid, (±)-DL-mandelic acid, methanesulphonic acid, naphthalene-2-sulphonic acid, naphthalene-2,5-disulphonic acid, 1-hydroxy-2-naphthalene-carboxylic acid, nicotinic acid, nitric acid, oleic acid, orotic acid (uracil-6-carboxylic acid), oxalic acid, palmitic acid, pamoa acid (embonic acid), phosphoric acid, L-pyroglutamic acid, salicylic acid, acetylsalicylic acid, 4-aminosalicylic acid, sebacic acid, stearic acid, succinic acid, sulphuric acid, tannic acid, (+)-L-tartaric acid, (±)-DL-tartaric acid, thiocyanic acid, p-toluenesulphonic acid and undecylenic acid. Preferred salts are hydrochloride, saccharinate, dihydrogen phosphate, hydrogen phosphate and phosphate. Particularly preferred is the hydrochloride salt, preferably of axomadol and of (+)-O-demethyl axomadol.

Axomadol or metabolites thereof can also be present as a mixture of salts of the above-mentioned organic and inorganic acids in any desired ratio.

In a preferred embodiment, axomadol or a metabolite thereof is present in a medicament. In yet another preferred embodiment, the medicament is a solid medicinal form. Liquid or pasty medicinal forms are also possible.

Preferably, the medicament is formulated for oral administration. However, pharmaceutical forms that are adapted for other administration routes are also possible, e.g. for buccal, sublingual, transmucosal, rectal, intralumbal, intraperitoneal, transdermal, intravenous, intramuscular, intragluteal, intracutaneous and subcutaneous application.

Depending on the formulation, the medicament preferably contains suitable additives and/or adjuvants. Suitable additives and/or adjuvants in the sense of the invention are all substances known to a person skilled in the art for the formation of galenic formulations. The choice of these adjuvants and also the quantities to be used are dependent on how the medicament is to be administered, i.e. orally, intravenously, intraperitoneally, intradermally, intramuscularly, intranasally, buccally or locally.

Preparations suitable for oral administration are those in the form of tablets, chewable tablets, lozenges, capsules, granules, drops, liquids or syrups, and those suitable for parenteral, topical and inhalatory administration are solutions, suspensions, easily reconstituted dry preparations and sprays. A further possibility is suppositories for rectal administration. The application in a depot in dissolved form, a patch or a plaster, possibly with the addition of agents promoting skin penetration, are examples of suitable percutaneous forms of application.

Examples of adjuvants and additives for oral forms of application are disintegrants, lubricants, binders, fillers, mould release agents, possibly solvents, flavourings, sugar, in particular carriers, diluents, colouring agents, antioxidants etc.

Waxes or fatty acid esters, amongst others, can be used for suppositories and carrier substances, preservatives, suspension aids etc. can be used for parenteral forms of application.

Adjuvants can be, for example: water, ethanol, 2-propanol, glycerine, ethylene glycol, propylene glycol, polyethylene glycol, polypropylene glycol, glucose, fructose, lactose, saccharose, dextrose, molasses, starch, modified starch, gelatine, sorbitol, inositol, mannitol, microcrystalline cellulose, methyl cellulose, carboxymethyl-cellulose, cellulose acetate, shellac, cetyl alcohol, polyvinylpyrrolidone, paraffins, waxes, natural and synthetic rubbers, acacia gum, alginates, dextran, saturated and unsaturated fatty acids, stearic acid, magnesium stearate, zinc stearate, glyceryl stearate, sodium lauryl sulphate, edible oils, sesame oil, coconut oil, peanut oil, soybean oil, lecithin, sodium lactate, polyoxyethylene and propylene fatty acid esters, sorbitane fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulphate, zinc sulphate, calcium sulphate, potash, calcium phosphate, dicalcium phosphate, potassium bromide, potassium iodide, talc, kaolin, pectin, crospovidon, agar and bentonite.

The production of these medicaments and pharmaceutical compositions is conducted using means, devices, methods and processes that are well known in the art of pharmaceutical technology, as described, for example, in “Remington's Pharmaceutical Sciences”, A. R. Gennaro, 17th ed., Mack Publishing Company, Easton, Pa. (1985), in particular in part 8, chapters 76 to 93.

Thus, for example, for a solid formulation such as a tablet, the active substance of the medicament can be granulated with a pharmaceutical carrier substance, e.g. conventional tablet constituents such as cornstarch, lactose, saccharose, sorbitol, talc, magnesium stearate, dicalcium phosphate or pharmaceutically acceptable rubbers, and pharmaceutical diluents such as water, for example, in order to form a solid composition that contains the active substance in a homogenous dispersion. Homogenous dispersion is understood here to mean that the active substance is uniformly dispersed throughout the composition, so that this can be readily divided into identically effective standard dose forms such as tablets, capsules, lozenges. The solid composition is then divided into standard dose forms. The tablets or pills can also be coated or otherwise compounded to prepare a slow release dose form. Suitable coating agents include polymeric acids and mixtures of polymeric acids with materials such as shellac, cetyl alcohol and/or cellulose acetate, for example.

In a preferred embodiment of the present invention axomadol or a metabolite thereof is present in the medicament in immediate release form. Such medicaments may be particularly useful for treating acute gastrointestinal cramps. The immediate release form, in particular the immediate release form comprising axomadol or (+)-O-demethyl axomadol is suitable for twice daily administration.

The medicament is preferably manufactured for administration twice daily (bid), or three times daily, the twice daily administration (bid) being particularly preferred.

In another preferred embodiment of the present invention axomadol or a metabolite thereof is present in the medicament in controlled-release form. Such medicaments may be particularly useful for treating chronic conditions.

The term controlled release as used herein refers to any type of release other than immediate release such as delayed release, prolonged release, sustained release, slow release, extended release and the like. These terms are well known to any person skilled in the art as are the means, devices, methods and processes for obtaining such type of release.

The medicament is preferably manufactured for administration once or twice daily (bid), the once daily administration (bid) being particularly preferred.

The quantities of axomadol to be administered to patients vary depending on the weight of the patient, the type of application and the severity of the illness. In a preferred embodiment, the medicament contains axomadol in a quantity of 10 to 2000 mg, more preferred 15 to 1000 mg, and still more preferred 20 to 500 mg, based on the free base.

Axomadol or a metabolite thereof can be released slowly from preparations that can be applied orally, rectally or percutaneously. The medicament is preferably manufactured for administration once daily (o.a.d.), twice daily (bid), or three times daily, the twice daily administration (bid) being particularly preferred.

A slow release of axomadol or a metabolite thereof can be achieved, for example, by retardation using a matrix, a coating or osmotically active release systems (cf. WO 2005/009329, for example).

In a preferred embodiment
the medicament is formulated for oral administration; and/or
the medicament is a solid and/or compressed and/or film-coated drug form; and/or
the medicament releases axomadol slowly from a matrix; and/or
the medicament contains axomadol in a quantity of 0.001 to 99.999% by wt., more preferred 0.1 to 99.9% by wt, still more preferred 1.0 to 99.0% by wt., even more preferred 2.5 to 80% by wt., most preferred 5.0 to 50% by wt. and in particular 7.5 to 40% by wt., based on the total weight of the medicament; and/or
the medicament contains a pharmaceutically compatible carrier and/or pharmaceutically compatible adjuvants; and/or
the medicament has a total mass in the range of 25 to 2000 mg, more preferred 50 to 1800 mg, still more preferred 60 to 1600 mg, more preferred 70 to 1400 mg, most preferred 80 to 1200 mg and in particular 100 to 1000 mg; and/or
the medicament is selected from the group comprising tablets, capsules, pellets and granules.

The medicament can be provided as a simple tablet and as a coated tablet (e.g. as film-coated tablet or lozenge). The tablets are usually round and biconvex, but oblong forms are also possible. Granules, spheres, pellets or microcapsules, which are contained in sachets or capsules or are compressed to form disintegrating tablets, are also possible. Medicaments containing at least 0.001 to 99.999% by wt. axomadol, in particular low effective doses, are preferred to avoid side-effects. The medicament preferably contains 0.01% by wt. to 99.99% by wt. axomadol, more preferred 0.1 to 90% by wt., still more preferred 0.5 to 80% by wt., most preferred 1.0 to 50% by wt. and in particular 5.0 to 20% by wt.

It is particularly preferred if the medicament is in a form for oral administration that is configured for twice daily application and contains axomadol in a quantity of 10 to 2000 mg based on the free base.

The medicament may contain one or more further drugs besides axomadol or a metabolite thereof. Preferably, however, the medicament contains axomadol as the only drug.

In a preferred embodiment, the medicament may contain a vitamin such as B1, B6, or B12, a probiotic such as Lactobacilli spp, or a prebiotic, or any mixture thereof. Suitable probiotics and prebiotics are disclosed for example in R. Spiller, Review article: probiotics and prebiotics in irritable bowel syndrome, Aliment Pharmacol Ther 28, 385-396.

In another one of its embodiments, the present invention relates to axomadol or a metabolite for use in a method for the treatment of irritable bowel syndrome.

In yet another one of its embodiments, the present invention relates to the use of axomadol or a metabolite thereof for the preparation of a medicament for the treatment of irritable bowel syndrome.

In yet another one of its embodiments, the present invention relates to a method for treating irritable bowel syndrome in a patient, preferably in a mammal, more preferably in a human, which comprises administering an effective and physiologically acceptable amount of axomadol or a metabolite thereof as described herein to a patient.

Preferably irritable bowel syndrome that also includes irritable colon is selected from the group consisting of irritable bowel syndrome with diarrhoea, diarrhea-predominant irritable bowel syndrome, irritable bowel syndrome without diarrhoea, constipation-predominant irritable bowel syndrome, irritable bowel syndrome with alternating stool pattern and post infectious irritable bowel syndrome.

Also preferably, irritable bowel syndrome is defined by ICD-10 (International Statistical Classification of Diseases and Related Health Problems, WHO edition, preferably version of 2007), i.e., it includes irritable colon [K58]. Preferably irritable bowel syndrome may include irritable bowel syndrome with diarrhoea [K58.0] and irritable bowel syndrome without diarrhea [K58.9]. Irritable bowel syndrome without diarrhoea may preferably also include irritable bowel syndrome not otherwise specified (NOS).

Even if the medicaments according to the invention exhibit few side effects only, it may be advantageous, for example, in order to avoid certain types of dependency to use morphine antagonists, in particular naloxone, naltrexone and/or levallorphan, in addition to axomadol or a metabolite thereof.

The present invention also relates to a kit comprising a medicament containing axomadol or a metabolite thereof (dosage forms) according to the invention.

The following gives a brief description of the figures:

FIG. 1 shows the effects of axomadol hydrochloride on the twitch contractions of the isolated guinea pig ileum.

Axomadol hydrochloride was applied cumulatively to electrically stimulated guinea pig ileum preparations. After the last application of axomadol hydrochloride, naloxone (10⁻⁶ M) was added. Drug effects on the twitch reaction were calculated as percentage of pre-value and expressed as mean±s.e.m. Four guinea pig ileum preparations per group were tested.

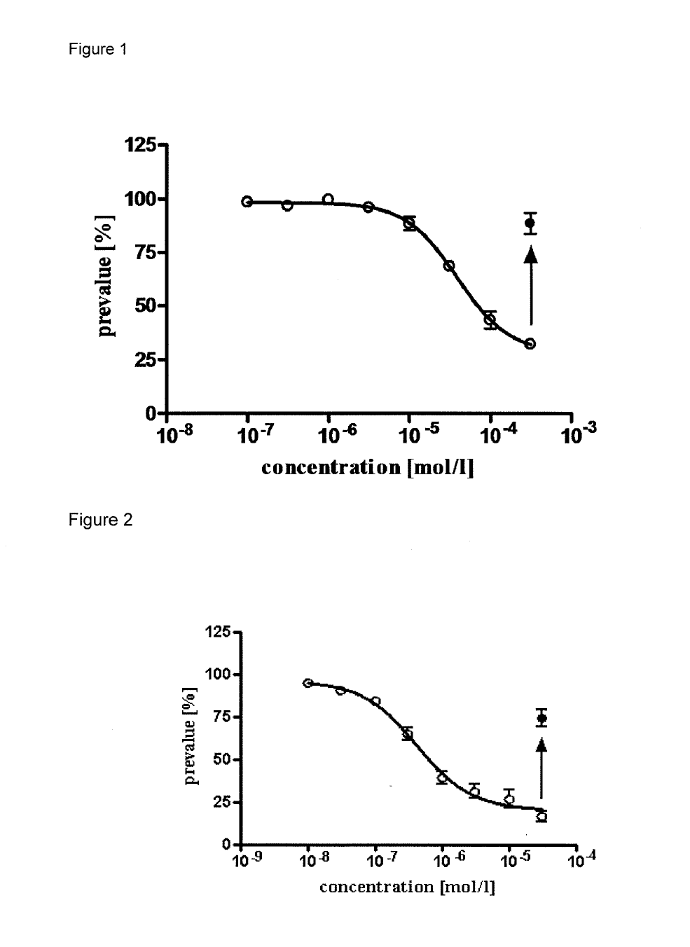

FIG. 2 shows the effects of (+)-O-demethyl axomadol hydrochloride on the twitch contractions of the isolated guinea pig ileum.

(+)-O-demethyl axomadol hydrochloride was applied cumulatively to electrically stimulated guinea pig ileum preparations. After the last application of axomadol hydrochloride, naloxone (10⁻⁶ M) was added. Drug effects on the twitch reaction were calculated as percentage of pre-value and expressed as mean±s.e.m. Four guinea pig ileum preparations per group were tested.

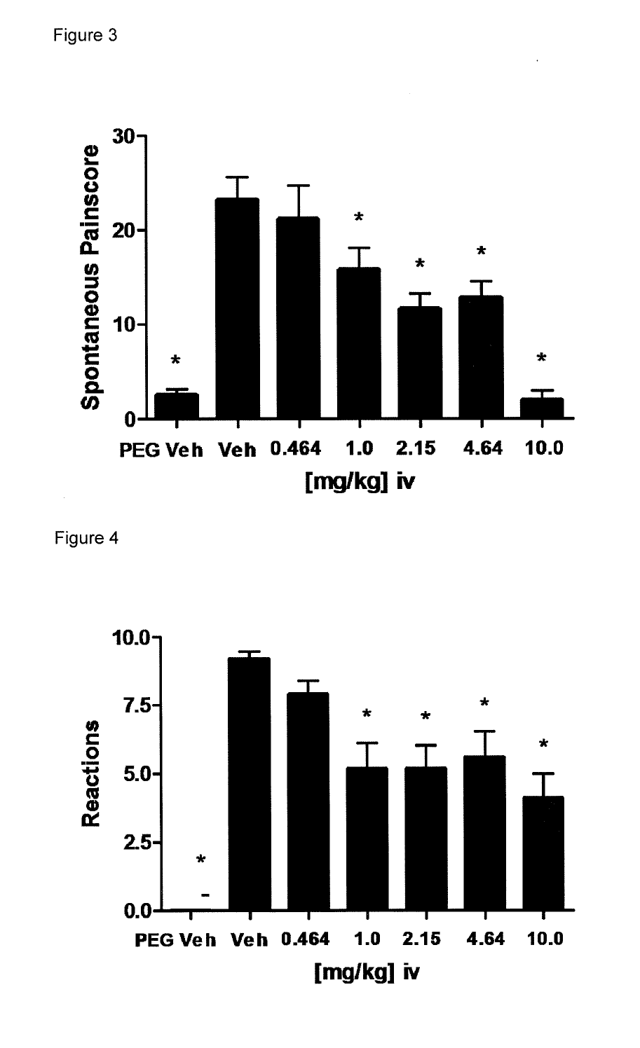

FIG. 3 shows the anti-nociceptive effect of axomadol hydrochloride ([mg/kg], i.v.) in mustard oil colitis, as measured as inhibition of the spontaneous pain score; * P<0.05 versus saline.

FIG. 4 shows the anti-allodynic effect of axomadol hydrochloride ([mg/kg], i.v.) in mustard oil colitis, as measured as inhibition of the referred allodynia; * P<0.05 versus saline.

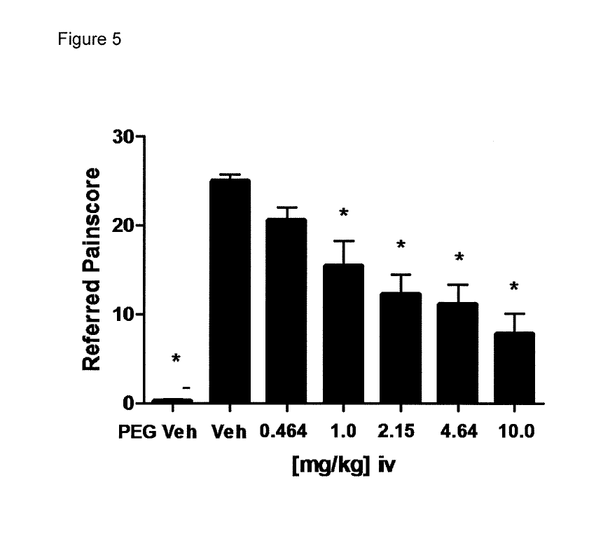

FIG. 5 shows the anti-hyperalgesic effect of axomadol hydrochloride ([mg/kg], i.v.) in mustard oil colitis, as measured as inhibition of the referred hyperalgesia; * P<0.05 versus saline.

In FIGS. 3, 4 and 5 PEG represents PEG200 [Polyethylene glycol; molecular weight 200 g/mol]; Veh represents vehicle solution 0.9% NaCl.

The following examples serve for a further explanation of the invention but should not be construed as restrictive.

The studies presented below clearly show the inhibitory effects of axomadol and its metabolite on ileum contractions and on visceral nociception, referred visceral hyperalgesia and allodynia. Thus, axomadol and its metabolite address major symptoms of IBS, abnormal gastrointestinal (GI) motility and visceral hypersensitivity and referred pain.

### EXAMPLES

### 1. Effects of Axomadol and (+)-O-Demethyl Axomadol on the Twitch Contractions of the Isolated Guinea Pig Ileum

It was investigated whether axomadol and (+)-O-demethyl axomadol are able to modulate gastrointestinal motility. For this purpose, the responses to these compounds were tested on electrically induced contractions of guinea pig ileum (so called Twitch reactions), which are known to be reduced e.g. by opioids (Paton, WDM. (1957) The action of morphine and related substances on contraction and on acetylcholine output of coaxially stimulated guinea-pig ileum. Br. J. Pharmacol. Chemother. 11: 119-127).

### 1.1 Experimental Animals

Male guinea pigs (PBW, Charles River, Kiβlegg, FRG) weighing 250-350 g were used for the study. The animals were kept under standard housing conditions: light/dark rhythm (06.00-18.00 h light, 18.00-6.00 h dark); room temperature 22±2° C., relative air humidity 55±5%; 15 air changes per hour, air movement <0.2 m/sec. The animals were given water and an exclusive diet of “Herilan RM 204” (Eggersmann Company, Rinteln/FRG) ad libitum. Before experimental preparation they were kept in groups of up to 5 animals in type IV Makrolon cages (Ebeco Company, Castrop-Rauxel, FRG). There were at least 4 days between delivery and testing.

### 1.2 Compounds

Axomadol hydrochloride and (+)-O-demethyl axomadol hydrochloride were dissolved in aqua bidest. Final concentrations in the organ bath ranged from 10⁻⁷ to 3.2·10⁻⁴ M and from 10⁻⁸ to 3.2·10⁻⁵ M for axomadol hydrochloride and (+)-O-demethyl axomadol hydrochloride respectively (cumulative drug application). Naloxone (10⁻⁶ M) was used as opioid antagonist.

### 1.3 Experimental Method

A four-compartment organ bath (Dept. Biotechnology, Grunenthal GmbH) consisting of 20 ml acrylic glass compartments, organ supports and force transducers (F10 force transducers, Type 375, HSE, FRG) was used for the measurement of isometric contractions. The organ bath was filled and emptied by means of a semi-automatic dosing arrangement. All experiments were performed at room temperature. The nutrient solution was gassed with carbogen (95% O_{2/5}% CO₂)) both in the nutrient storage chamber and in the organ bath starting 30 min before the beginning and throughout the experiment.

The nutrient solution had the following composition:

| | |
|---|---|
| NaCl | 118.0 mM |
| KCl | 4.8 mM |
| CaCl₂•2H₂O | 1.3 mM |
| KH₂PO₄ | 1.2 mM |
| MgSO₄•7H₂O | 1.2 mM |
| NaHCO₃ | 25.0 mM |
| Glucose | 11.0 mM |
| Ascorbic acid | 0.57 mM |
| Na₂-EDTA | 0.03 mM |
| (pH: 7.4-7.5) | |

Parameter: contraction force [g]

### 1.4 Experimental Performance

Guinea pigs were killed in CO₂-atmosphere and the ileum was dissected free from adhering tissue, removed and suspended in the organ bath. After an incubation period of at least 30 min, isometric contractions (twitch reactions) were elicited by transmural stimulation pulses (duration 1 ms, amplitude 180 mA at 0.03 Hz; stimulator A310, WPI, FRG). A pre-tension of 1 g was applied to the preparations and constantly readjusted during the equilibration period (at least 30 minutes) while the nutrient was changed twice.

After having registered the pre-value, test compounds were added to the organ bath in cumulative concentration steps as indicated. The exposure time for each concentration was 6 minutes. After the last application of the test compound, the opioid-antagonist naloxone (10⁻⁶ M) was added without previous wash out of the test compound.

1.5 Evaluation Data was calculated as the mean electrically stimulated contraction force during a period of 4 to 6 minutes after compound application and expressed as percentage of the pre-value. The mean contraction force during a period of 2 minutes before compound application was taken as pre-value. All results were expressed as means±s.e.m. of ≧4 single experiments. For determination of IC₅₀ values, regression lines (y=f log x) were constructed and IC₅₀ values with s.e.m. were calculated using a computer-assisted regression analysis program (Grünenthal GmbH). The reversal of the test compound's activity by the antagonist was determined according to the following equation:

$$\% reversal = 100 \% - \frac{max . {effect}_{test compound + antagonist}}{max . {effect}_{test compound}} \times 100 \%$$

with max. effect being:
100%−% reduction of pre-value of twitch reaction at highest dose of test compound.

### 1.6 Results

Axomadol hydrochloride reduced the electrically induced contractions of the isolated guinea pig ileum in a concentration-dependent manner with a threshold of around 10 μM and an IC₅₀ value of 39.2±2.8 μM (see FIG. 1 and Table 1). With increasing concentrations of axomadol up to 320 μM, the twitch reactions were markedly reduced (reduction to 32.1% of pre-value). The inhibitory effect of axomadol hydrochloride was reversed by 83.4% after addition of the opioid receptor antagonist naloxone (1 μM; see FIG. 1, Table 1).

(+)-O-demethyl axomadol hydrochloride reduced the electrically induced contractions of the isolated guinea pig ileum in a concentration-dependent manner with a threshold of around 0.032 μM and an IC₅₀ value of 0.43±0.09 μM (see FIG. 2 and Table 1). With increasing concentrations of (+)-O-demethyl axomadol hydrochloride up to 32 μM, the twitch reactions were markedly reduced (reduction to 16.6±3% of pre-value). The inhibitory effect of (−)-O-demethyl axomadol hydrochloride was reversed by 69.1% after addition of the opioid receptor antagonist naloxone (1 μM; see FIG. 2, Table 1).

TABLE 1
Effects of axomadol hydrochloride and
(+)-O-demethyl axomadol hydrochloride on
twitch contractions of the guinea pig ileum

| | IC₅₀ | maximal reduction | % reversal |
|---|---|---|---|
| | [10⁻⁶M] | [% pre-value] | by naloxone |
| axomadol | 39.2 ± 2.8 | 32.1 | 83.4 |
| hydrochloride | | | |
| (+)-O-demethyl | 0.43 ± 0.09 | 16.6 | 69.1 |
| axomadol | | | |

### 2. Effects of Axomadol on Visceral Hyperalgesia

The effects of axomadol hydrochloride on visceral hyperalgesia were studied, which was induced by rectal administration of mustard oil in mice (according to Laird J M, Martinez-Caro L, Garcia-Nicas E, Cervero F. (2001) A new model of visceral pain and referred hyperalgesia in the mouse. Pain 92: 335-42). The typical visceral pain behaviour was quantified in three parameters: During the first minutes after mustard oil administration spontaneous visceral pain behaviour occurs. Following this period of spontaneous pain, referred allodynia and hyperalgesia can be quantified by means of von Frey filaments of different strength stimulating the abdomen of the mice.

### 2.1 Animals

Male NMRI mice (28-38 g body weight) from a commercial breeder (Iffa Credo, France) were used. The animals were housed under standardized conditions: light/dark cycle (06.00-18.00 h light, 18.00-06.00 h dark), room temperature 20-24° C., relative air humidity 35-70%, 15 air changes per hour, air movement <0.2 m/sec, tap water and standard diet ad libitum, macrolon type 4 cages with maximally 30 animals per cage. There were at least 5 days between delivery and start of the experiment.

### 2.2 Compounds

Axomadol hydrochloride was dissolved in vehicle solution and injected intra-venously.

Doses: 0.464; 1.0; 2.15; 4.64; 10.0 mg/kg i.v.
Administration volume: 10 ml/kg
Vehicle solution: 0.9% NaCl solution (Fresenius, Bad Homburg, FRG)
Mustard oil was dissolved in vehicle solution and administered intra-rectally
Dose: 50 μl of a 3.5 Vol.−% solution per animal
Vehicle solution: PEG 200

### 2.3 Experimental Preparation

2.3.1 Induction of Colitis Animals were habituated to the test conditions for 20-30 min and stimulated with von Frey filaments onto the abdominal wall. 10 stimulations with von Frey filaments of 1, 4, 8, 16, and 32 mN were applied in ascending order (i.e. 10×1 mN, 10×4 mN, etc.). Animals with more than 25 positive reactions during this phase were excluded. Vaseline was applied in the perianal area to avoid the stimulation of somatic areas with the irritant chemical. Colitis is induced by rectal administration of 50 μl mustard oil (3.5%). Control animals are treated with vehicle (50 μl PEG200).

### 2.3.2 Prophylactic Treatment

Axomadol hydrochloride or vehicle was given intravenously (i.v.) 5 min before mustard oil. Seven animals were tested per group. The following parameters were counted:

2-12 min after mustard oil:
1. Spontaneous pain score: counting and scoring of visceral pain behaviours (Score 1-2, 1=licking of abdominal wall, 2=stretching, squashing, mounting, backward-movement or contraction of the flank muscles).
   20-40 min after mustard oil
2. Referred allodynia (number of reactions): counting of withdrawal reactions against 10 stimulations with a 1 mN von Frey filament.
3. Referred hyperalqesia (referred pain score): counting and scoring of withdrawal reactions against 10 stimulations with a 16 mN von Frey filament (Score 1-3, 1=lifting of abdomen, licking, movement, 2=extrusion or flinching of hind paws, slight jumping, strong licking, 3=strong jumping, vocalisation).

### 2.4 Statistical Analysis

Data were analysed by means of two-factor analysis of variance (ANOVA) with repeated measures. Significance of treatment-, time- or treatment x time interaction effects was analyzed by means of Wilks' Lambda statistics. In case of a significant treatment effect, pair-wise comparison was performed at the every test time point on raw data by Fisher's least significant difference test. Results were considered statistically significant if P<0.05. ED₅₀ values and 95% confidence intervals was calculated by linear regression.

### 2.5 Results

Axomadol hydrochloride was tested in doses of 0.464; 1.0; 2.15; 4.64 and 10.0 mg/kg i.v. and showed a dose dependent and significant inhibition of all three visceral pain parameters. Spontaneous visceral pain behaviour (FIG. 3), referred allodynia (FIG. 4) and referred hyperalgesia (FIG. 5) were inhibited with ED₅₀-values (95% confidence intervals) of 2.62 (1.79-4.05)/6.13 (5.56-6.60-4.75) and 2.72 (1.58-5.46) mg/kg i.v., respectively.

1. Axomadol or a metabolite thereof for use in the treatment of irritable bowel syndrome.

2. Axomadol or a metabolite thereof according to claim 1, wherein the metabolite is selected from the group consisting of O-demethyl axomadol, N-demethyl axomadol and N,O-didemethyl axomadol.

3. Axomadol or a metabolite thereof according to claim 1, wherein it is present in a medicament.

4. Axomadol or a metabolite thereof according to claim 3, wherein the medicament is solid.

5. Axomadol or a metabolite thereof according to claim 3, wherein the medicament is formulated for oral administration.

6. Axomadol or a metabolite thereof according to claim 3, wherein the medicament is a tablet.

7. Axomadol or a metabolite thereof according to claim 3, wherein the medicament is formulated for administration twice daily (bid).

8. Axomadol or a metabolite thereof according to claim 3, wherein the medicament contains axomadol in an amount of 10 to 1000 mg.

9. Metabolites of axomadol selected from the group consisting of (±)-N-demethyl axomadol, (+)-N-demethyl axomadol, (−)-N-demethyl axomadol, (±)-N,O-didemethyl axomadol, (+)-N,O-didemethyl axomadol and (−)-N,O-didemethyl axomadol.

10. Axomadol or a metabolite thereof according to claim 2, wherein it is present in a medicament.

11. Axomadol or a metabolite thereof according to claim 4, wherein the medicament is formulated for oral administration.

12. Axomadol or a metabolite thereof according to claim 4, wherein the medicament is a tablet.

13. Axomadol or a metabolite thereof according to claim 5, wherein the medicament is a tablet.

14. Axomadol or a metabolite thereof according to claim 4, wherein the medicament is formulated for administration twice daily (bid).

15. Axomadol or a metabolite thereof according to claim 5, wherein the medicament is formulated for administration twice daily (bid).

16. Axomadol or a metabolite thereof according to claim 6, wherein the medicament is formulated for administration twice daily (bid).

17. Axomadol or a metabolite thereof according to claim 4, wherein the medicament contains axomadol in an amount of 10 to 1000 mg.

18. Axomadol or a metabolite thereof according to claim 5, wherein the medicament contains axomadol in an amount of 10 to 1000 mg.

19. Axomadol or a metabolite thereof according to claim 6, wherein the medicament contains axomadol in an amount of 10 to 1000 mg.

20. Axomadol or a metabolite thereof according to claim 7, wherein the medicament contains axomadol in an amount of 10 to 1000 mg.
